(19) United States
(12) Patent Application Publication
(10) Pub. No.: US 2026/0229330 A1
(43) Pub. Date: Aug. 6, 2026

(54) Generative AI Powered Prior Authorization Decision Framework

(71) Applicant: Cerner Innovation, Inc., Kansas City, MO (US)

(72) Inventors: Suman Pal, Bangalore (IN); Pragnya Ranjan Pradhan, Similipada (IN); Shanthan Kesharaju, Cary, NC (US)

(73) Assignee: Cerner Innovation, Inc., Kansas City, MO (US)

(21) Appl. No.: 19/322,374

(22) Filed: Sep. 8, 2025

Related U.S. Application Data
(60) Provisional application No. 63/753,133, filed on Feb. 3, 2025.

(51) Int. Cl. G16H 15/00; G06F 40/205; G16H 20/00

(52) U.S. Cl. CPC G16H 15/00; G06F 40/205; G16H 20/00

(57) ABSTRACT

Techniques for leveraging generative AI in generating prior authorization reports for procedure requests for patients are disclosed. Initially, the system accesses clinical guidelines associated with an insurer of a patient. The system generates a query vector embedding for a request query for the procedure and vector embeddings for the clinical guidelines. Based on a similarity search of the vector embeddings, the system identifies relevant clinical guidelines. The system then provides a prompt to a generative AI model to generate a prior authorization report responsive to the procedure request. The generative AI model is applied to the relevant clinical guideline and patient data, e.g., the request query, health plan details, and patient details, to generate the prior authorization report. The system generates a report based on a target clinical guideline and the patient's data. The prior authorization report provides information including approval or denial of the procedure.

### BENEFIT CLAIMS; RELATED APPLICATIONS; INCORPORATION BY REFERENCE

This application claims the benefit of U.S. Provisional Patent Application 63/753,133, filed Feb. 3, 2025, which is hereby incorporated by reference.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to machine learning applications in automated healthcare systems. In particular, the present disclosure relates to leveraging generative AI in prior authorization decision frameworks.

### BACKGROUND

Prior authorization is a process used in healthcare where a patient or their healthcare provider is required to obtain approval from a patient's health insurance company before receiving a specific treatment, medication, or procedure. Prior authorization ensures that the treatment is covered by the insurance and is deemed medically necessary based on insurer guidelines.

Automating prior authorization in healthcare faces significant technical challenges despite being a high-value use case for interoperability and artificial intelligence applications. Healthcare data is fragmented across disparate electronic health record (EHR) systems, payer platforms, and pharmacy systems, many of which utilize different data standards or rely on unstructured formats such as free-text physician notes or scanned documents. Even where interoperability standards exist, adoption is inconsistent and incomplete.

Other challenges arise from rule heterogeneity and system integration. Different entities may maintain their own authorization rules, forms, and criteria, which are subject to frequent change and may not be published in machine-readable formats. Thus, automated systems must be able to adapt to opaque and dynamic rules. As a result, any hard-coding of such rules into software systems must be repeatedly reconfigured or patched, which may create software instability, frequent downtime, and inconsistent execution of rule-based logic across systems. Poorly designed prior authorization automation may also disrupt workflows, introduce redundant alerts, and/or cause increased system inefficiencies instead of reductions.

The approaches described in this section are approaches that could be pursued but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a prior authorization decision support system in accordance with one or more embodiments;

FIG. 2 illustrates an example set of operations for leveraging generative AI in generating prior authorization reports for medical procedures for patients in accordance with one or more embodiments;

FIG. 3 illustrates an example set of operations for determining relevant clinical guidelines for prior authorization requests for patients in accordance with one or more embodiments;

FIG. 4A illustrates an example request interface for a prior authorization request system;

FIG. 4B illustrates an example prior authorization report interface for a prior authorization request system;

FIG. 5A illustrates a machine learning (ML) engine in accordance with one or more embodiments;

FIG. 5B illustrates an example set of operations of an ML engine in accordance with one or more embodiments; and

FIG. 6 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. PRIOR AUTHORIZATION DECISION SUPPORT SYSTEM ARCHITECTURE
3. LEVERARGING GENERATIVE AI IN GENERATING PRIOR AUTHORIZATION REPORTS FOR MEDICAL PROCEDURES FOR PATIENTS
4. DETERMINING RELEVANT CLINICAL GUIDELINES FOR PRIOR AUTHORIZATION REQUESTS
5. EXAMPLE PRIOR AUTHORIZATION REQUEST INTERFACE AND PRIOR AUTHORIZATION REPORT
6. PRACTICAL APPLICATIONS, ADVANTAGES & IMPROVEMENTS
7. MACHINE LEARNING ARCHITECTURE
8. MACHINE LEARNING ENGINE OPERATIONS
9. GENERATIVE AI MODELS
10. HARDWARE OVERVIEW
11. MISCELLANEOUS; EXTENSIONS

### 1. GENERAL OVERVIEW

One or more embodiments leverage generative AI to automatically generate a prior authorization report for a medical procedure request for a patient. Initially, the system accesses clinical guidelines associated with an insurer of the patient. The system generates a query vector embedding for a request query for a medical procedure for the patient and vector embeddings for the clinical guidelines. Based on a similarity search of the vector embeddings, the system identifies relevant clinical guidelines. The system provides a prompt to a generative AI model for generating a prior authorization report in response to receiving a prior authorization request for the medical procedure. The prompted generative AI model is applied to the relevant clinical guidelines and patient data to determine a target clinical guideline and generate a prior authorization report. The patient data may include the request query, health plan details, and patient details. The system generates a prior authorization report based on the target clinical guideline and the patient data. The prior authorization report provides information that includes approval or denial of the procedure.

One or more embodiments generate the vector embedding for the clinical guidelines by parsing the clinical guidelines to identify headings and subheadings. The system then segments the clinical guidelines into subsets or chunks based on the headings and subheadings. Chunking the clinical guidelines into small, semantically coherent units improves retrieval precision/recall, reduces latency and memory, fits LLM context limits, and enables parallel processing, caching, and incremental re-indexing. The system may use similarity searches (e.g., cosine similarity and/or reranking models) to identify the relevant clinical guideline(s).

One or more embodiments described in this Specification and/or recited in the claims may not be included in this General Overview section.

### 2. PRIOR AUTHORIZATION DECISION SUPPORT SYSTEM ARCHITECTURE

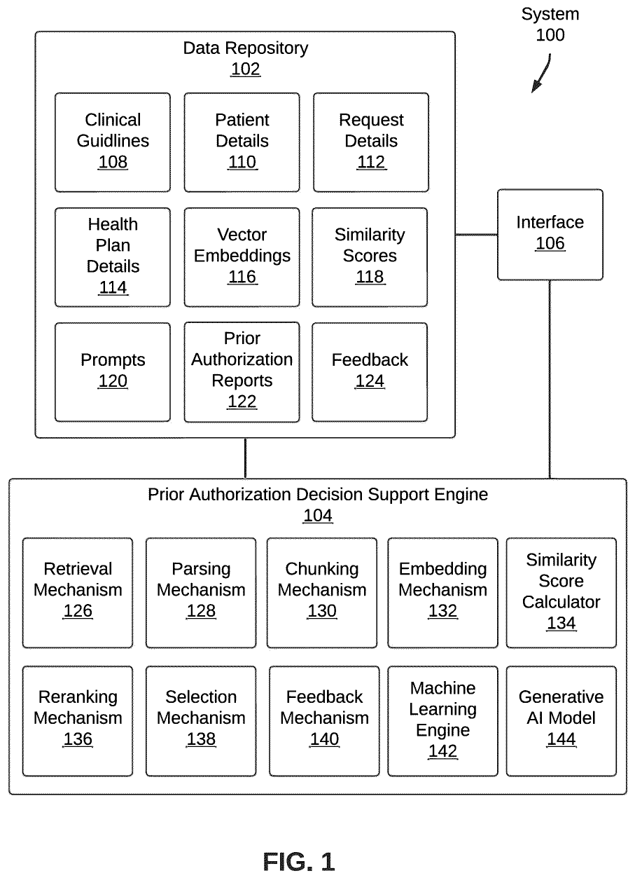

FIG. 1 illustrates a prior authorization decision system 100 in accordance with one or more embodiments. As illustrated in FIG. 1, system 100 includes a data repository 102, a prior authorization decision support engine 104, and an interface 106. In one or more embodiments, system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, data repository 102 is any type of storage unit and/or device (e.g., a file system, database, collection of tables, or any other storage mechanism) for storing data. Furthermore, data repository 102 may include multiple different storage units and/or devices. The multiple different storage units and/or devices may or may not be of the same type or located at the same physical site. Furthermore, data repository 102 may be implemented or executed on the same computing system as prior authorization decision support engine 104. Additionally, or alternatively, a data repository 102 may be implemented or executed on a computing system separate from prior authorization decision support engine 104. Data repository 102 may be communicatively coupled to prior authorization decision support engine 104 via a direct connection or via a network.

Information describing leveraging generative AI to automatically generate prior authorization reports for medical procedure requests for patients may be implemented across any of components within the system 100. However, this information is illustrated within the data repository 102 for purposes of clarity and explanation.

In one or more embodiments, data repository 102 includes clinical guidelines 108, patient details 110, request details 112, health plan details 114, vector embeddings 116, similarity scores 118, prompts 120, prior authorization reports 122, and feedback 124.

In one or more embodiments, clinical guidelines 108, also known as payer medical policies, coverage criteria, or utilization-management guidelines, are guidelines created by health insurance providers, e.g., insurance companies, for determining whether or not to authorize a medical procedure for a patient, e.g., an insured individual. Clinical guidelines may be particular to an insurance provider, organization, or governing body. Clinical guidelines for the same insurer may vary based on geography, e.g., from state to state and/or from country to country. Clinical guidelines provided by the Centers for Medicare & Medicaid Services (CMS) include National Coverage Determination (NCD) and Local Coverage Determination (LCD). NCDs are clinical guidelines that provide for decisions made at the national level, and they apply to Medicare beneficiaries across the United States. LCDs provide for decisions made by Medicare Administrative Contactors that administer Medicare claims and benefits in specific geographic regions or jurisdictions.

In one or more embodiments, clinical guidelines 108 are particular to an insurance provider. Clinical guidelines may include eligibility criteria, approved treatments, required documentation, treatment protocols, and exclusions. Clinical guidelines may be accessed through insurance company websites and provider portals. Insurance companies may provide clinical policy information and coverage guidelines on their websites. Web scraping may be implemented to access the information from the websites. Insurance providers may provide dedicated portals for accessing clinical guidelines and coverage criteria. Insurance providers may offer application programming interfaces (APIs) that enable secure access to databases including clinical guidelines and coverage information.

In one or more embodiments, clinical guidelines 108 are included in resources provided at Brighton Health Plan Solutions Policies website and are available in PDF format.

In one or more embodiments, patient details 110 refer to a record of clinically relevant information associated with a patient. Patient details may be keyed by time, encounter, and/or source. Patient details 110 may include demographics and identifiers, clinical notes, diagnostic results, active problem list and diagnoses, medications and allergies, procedures and immunizations, vital signs and measurements, social and behavioral determinants, and/or family medical history.

In one or more embodiments, clinical notes include history and physical exams, progress notes, consultations, operative and discharge summaries, and/or secure messages. Clinical notes may include structure or unstructured text that identify problems, symptoms, physical findings, assessments/plans, procedures, medications, allergies, vitals, and social factors. Clinical notes may include terminologies, e.g., SNOMED CT, ICD-10-CM, CPT/HCPCS, and RxNorm.

In one or more embodiments, diagnostic results include laboratory and imaging data. Diagnostic results may include order or test identifiers, measured values and Unified Code for Units of measure (UCUM) units, reference ranges, abnormal flags, specimen and collection metadata, device identifiers, and/or interpretive statements. Observations associated with diagnostic results may include modality, body site, technique, structured findings, and impressions. Diagnostic results may be temporally indexed. Temporal indexing of diagnostic results may support longitudinal queries, e.g., the last three HBA1C values within 180 days.

In one or more embodiments, family medical history refers to a structured dataset that encodes health conditions observed in an individual's biological relatives. In a family medical history, the system may represent a relative as a node with attributes comprising relationship type and degree (e.g., mother, father, sibling, second-degree), lineage side (maternal/paternal), biologic/adoptive status, twin/consanguinity flags, and vital status. The system may link each node to one or more condition records including standardized codes (e.g., SNOMED CT/ICD-10-CM), age or period of onset, severity, outcome (e.g., cause of death), and/or genetic findings (e.g., gene/variant identifiers) and risk modifiers. Negative assertions (e.g., “no known family history of colorectal cancer”) may be captured explicitly with assertion type and observation date. The nodes may carry provenance (source document or intake form, clinician/author, collection method), timestamps, and extraction confidence, and nodes are normalized to interoperable resources (e.g., HL7 FHIR FamilyMemberHistory with linked Condition/Observation elements) to support deterministic queries. A reconciliation process may merge duplicate or conflicting reports across sources (electronic health record (EHR) notes, patient-reported forms, genetic reports) using recency, source reliability, and concept equivalence rules.

In one or more embodiments, request details 112 refer to various information, including a description of the procedure or service being requested, a diagnosis related to the procedure, the medical necessity for the procedure, a proposed date of the procedure, and/or any alternative treatments considered or attempted. The description of the procedure may include a Current Procedural Terminology (CPT) code. CPT codes are standardized codes used to describe medical, surgical, and diagnostic procedures. The diagnosis related to the procedure may be accompanied by an International Classification of Diseases (ICD) code. Request details relating to medical necessity for the procedure may include clinical notes, patient history, and any previous treatments relevant to the request. The proposed date of the procedure may also include a location where the procedure is to be performed. The alternative treatments considered or attempted may assist in justifying the need for the requested procedure. When the request details are included in the EHR of the patient, the system may access the request details directly. Alternatively, a healthcare professional may manually enter the request detail into the respective fields of the prior authorization request interface.

In one or more embodiments, location of service details includes a facility name, a facility address, facility contact information, department or specific area with the facility, a provider at the location, a type of facility, a national provider identifier (NPI), and a tax identification number (TIN). Facility name includes the hospital, clinic, or medical office where the procedure will be carried out. Facility address is important for verifying whether a facility is in-network or out-of-network. Location of service details may include identifying a specialized department, e.g., radiology, cardiology, or an outpatient surgery center within a hospital. Types of facilities include hospitals, outpatient surgery centers, specialty clinics, or other types of healthcare facilities. The type of facility may impact coverage details, for some insurance plans have different coverage rules for different types of facilities. NPI and TIM may be used to confirm that the services are proved by registered healthcare professionals and facilities. Location of service details may be accessed and entered into a prior authorization request interface in the same or similar manner as patient details and request details.

In one or more embodiments, health plan details 114 refer to various information for a patient, including covered services and procedures, exclusions and limitations, network status, deductibles and copayments, benefit maximums, and policy effective dates. Health plan details may be accessed by the system in the same manner as the clinical guidelines. Additionally, health plan details may be available in an EHR of the patient and/or by contacting the insurance company directly. Benefit design elements may include service-category and code-level coverage flags, site-of-care restrictions, frequency limits, referral requirements, prior-authorization requirements and exemptions, step-therapy programs, quantity/age/gender edits, place-of-service constraints, and/or medical-necessity policy references with effective versions. Pharmacy sections may include formulary identifier/version, tiering, prior-auth/step-therapy/quantity-limit tables, and/or specialty-pharmacy channel requirements. Financial elements may include network tiering, deductible/coinsurance/copay schedules, remaining accumulators (deductible, out-of-pocket max), visit-count accumulators (e.g., PT/OT), and benefit maximums.

In one or more embodiments, vector embeddings 116 refer to text that has been converted to a numeric format. The vector embeddings 116 are representations of individual words for text analysis, typically in the form of a real-valued vector. Vector embeddings 116 may represent individual text items or an aggregation of text items. Vector embeddings 116 may be formed using various word embedding techniques. Vector embeddings 116 may represent (i) clinical guidelines and/or (ii) request details.

In one or more embodiments, similarity scores 118 refer to an indication of the similarity between vector embeddings 116. The higher a similarity score, for example, the closer to 1.0, depending on the scale, the greater a semantic match between vector embeddings 116, e.g., between a vector embedding for a clinical guideline and a vector embedding for a request query. Similarity scores 118 may be weighted to reflect the relevance of the type of data used to calculate vector embeddings 116.

In one or more embodiments, prompts 120 refer to a text input that the generative AI model uses to generate a response, e.g., an authorization report. Prompts 120 set the context or task for the model. Prompts 120 are used to specify the task, constraints, grounding facts, and required output format for producing an authorization report. The prompt may include specific instructions, context information, or styled text to guide the desired output. Prompts 120 may include i) role assignment, ii) inputs, iii) output format, and/or iv) reasoning steps. Role assignment may include having the generative AI model respond as a medical expert evaluating requests for appropriateness and cost-effectiveness of a medical procedure. Inputs may include clinical notes, family history, diagnostic results, procedure requests, health plan details, CPT and ICD descriptions, supporting documents, and location of service details. Output format may include a decision, a table, recommendations, and a guideline source. Decisions may include approved, step therapy, alternative treatment, denied. A table summarizes symptoms, guidelines alignment, health plan coverage, and service location. Recommendations may include suggested alternatives. The guideline source may include citations to PDF references. Reasoning steps may include instructions to (a) evaluate clinical symptoms, family medical history, and diagnostic results, (b) match symptoms and request with guidelines, (c) assess an appropriateness of requested procedure for the location of service, and (d) verify compatibility with health plan coverage and alignment.

In one or more embodiments, authorization reports 122 refer to reports that summarize and evidence prior-authorization evaluations. Authorization reports 122 may include submission metadata, request details, patient details bound to evaluated criteria, health plan details in force, a target clinical guideline, and/or final disposition, i.e., decision. Submission metadata may include request ID, timestamps, ordering/rendering/facility NPIs, and/or place of service. A decision may include approve/pend/deny. Authorization reports 122 may include rationale, mapped reason codes, counterfactual recommendations, and/or any subsequent appeal or peer-to-peer outcomes.

In one or more embodiments, authorization reports 122 are output by generative AI model 144. The output format of authorization reports 122 may include (i) a decision, (ii) one or more tables, (iii) one or more recommendations, and/or (iv) a guideline source. A decision may include indication of approval or denial, step therapy, or alternative treatment. Tables may include a summary of symptoms, guidelines alignment, health plan coverage, and/or service location. Recommendations provided in authorization reports 122 may suggest alternatives to the requested procedure.

In one or more embodiments, feedback 124 refers to information captured before, during, and after prior-authorization adjudication used to correct case-level outcomes and continuously improve guideline execution, extraction models, and/or routing logic. Feedback 124 may include event type (e.g., reviewer override, appeal outcome, peer-to-peer note, provider correction, claims adjudication result, duplicate/withdrawal flag), scope (decision-level, criterion-level, or extractor-level), payload (structured reason codes and free-text annotations with bound codes/values/units and, for notes, character-offset spans), actor and role, timestamps, provenance (submission channel, document IDs), guideline version, plan configuration, data snapshot, and/or model parameters in force. Feedback 124 may include determinations and annotations from subject matter experts, provider portal responses and attachments, appeal decisions, post-service claims outcomes, and/or automated validators (e.g., contradiction checks, service level agreements (SLA) violations, drift detectors).

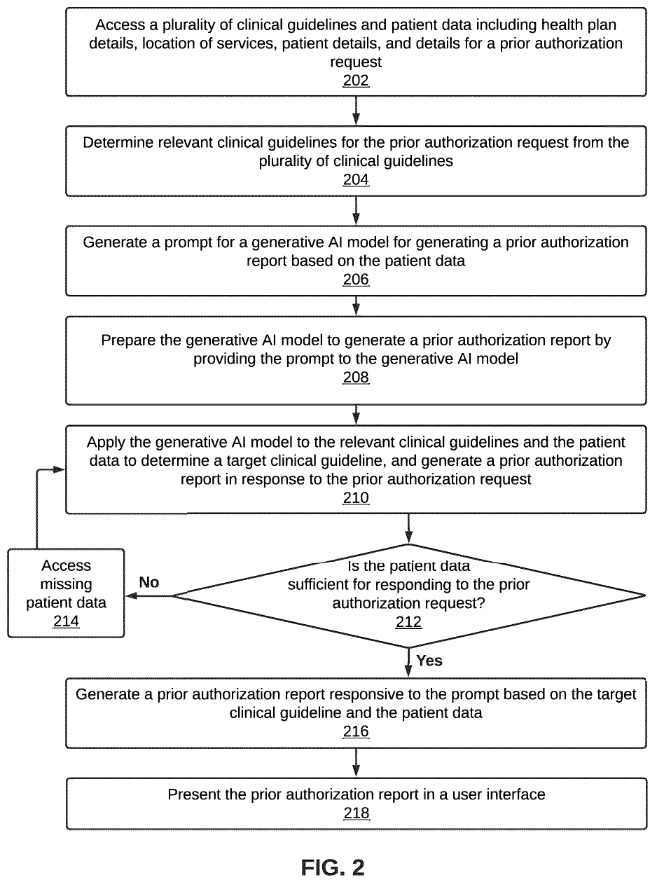

In one or more embodiments, prior authorization decision support engine 104 refers to hardware and/or software configured to perform operations described herein for leveraging generative AI to generate prior authorization reports for medical procedures for patients; examples are described below with reference to FIG. 2.

In an embodiment, prior authorization decision support engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, prior authorization decision support engine 104 includes a retrieval mechanism 126. Retrieval mechanism 126 refers to hardware and/or software configured to perform operations described herein for accessing and retrieving information from data sources. This may include database retrieval and information retrieval. In relational databases, Structured Query Language (SQL) is used for querying and retrieving data. SQL allows for specifying exactly what data to retrieve and how it should be processed. In Not Only SQL (NoSQL) databases, retrieval mechanisms may vary significantly depending on the type of NoSQL database, e.g., document, key-value, graph, or column store. These might use simpler, more scalable retrieval methods suited to the specific data model. Information retrieval is used primarily in search engines and large content management systems designed to handle unstructured data like text. Techniques include keyword search, semantic search, and full-text search. Retrieval mechanism 126 may use APIs to access data. Retrieval mechanism 126 may employ web scrapping techniques to acquire data from websites.

In one or more embodiments, prior authorization decision support engine 104 includes a parsing mechanism 128. Parsing mechanism 128 refers to hardware and/or software configured to perform operations described herein for ingesting, analyzing, and/or structurally normalizing clinical guidelines. Parsing mechanism 128 may accept heterogeneous inputs, e.g., web pages (HTML), downloadable documents (PDF, DOCX), semi-structured tables (CSV/XLSX), and scanned images. Parsing mechanism 128 may apply optical character recognition (OCR) and page-layout analysis to recover headings, lists, tables, footnotes, and cross-references. Parsing mechanism 128 may include a sectionizer that detects canonical policy regions (e.g., title/identifier, issuing organization, effective/review dates, product line and care-setting scope, “When Covered/When Not Covered,” “Policy Guidelines,” “Documentation Requirements,” and “Billing/Coding”) and anchors each to page/paragraph coordinates. Tokenization, pattern grammars (regular expressions, PEG/ANTLR rules), and ontology-assisted natural language processing (NLP) may extract (i) structured fields, including covered and non-covered indications expressed as Boolean/numeric/temporal conditions; (ii) prerequisite pathways (e.g., step therapy with permitted substitutions and bypass rules); (iii) contraindications and safety exceptions; (iv) site-of-care and frequency limits; (v) documentation checklists; and/or (vi) code bindings (CPT/HCPCS, ICD-10-CM/PCS, LOINC with UCUM units, RxNorm, place-of-service). Parsing mechanism 128 may normalize units to UCUM, validate code syntax and ranges, resolve synonyms via controlled vocabularies (e.g., SNOMED CT mappings), and/or convert narrative criteria into executable predicates with parameters (thresholds, look-back windows, counts, before/after relations).

In one or more embodiments, prior authorization decision support engine 104 includes a chunking mechanism 130. Chunking mechanism 130 refers to hardware and/or software configured to perform operations described herein for segmenting inputs into machine-interpretable chunks. Chunking mechanism 130 may optimize chunks for retrieval, embedding, and/or deterministic evaluation. Chunking mechanism 130 may accept structured, semi-structured, and unstructured sources (e.g., HTML/PDF/DOCX/CSV/XLSX, OCR scans). Chunking mechanism 130 may produce a sequence of versioned chunk records. A chunk record may include a stable chunk identifier, normalized text payload, layout metadata (page/section/heading path, bounding boxes for PDF regions, table cell coordinates), language and charset, token and character counts, boundary lineage (predecessor/successor chunk IDs, source offsets), provenance (URI, file hash, parser configuration/model versions), and/or policy/effective-date scope where applicable.

In one or more embodiments, chunking mechanism 130 employs multi-strategy segmentation, including the following: (i) structural chunking that honors headings, lists, tables, and code blocks while enforcing “keep-with-next” rules to avoid orphaned captions or fragmented tables; (ii) semantic chunking that uses topic-shift detectors or transformer-based segmenters to align boundaries with concept changes; and/or (iii) token-budget chunking that ensures each chunk fits target context/embedding limits with configurable overlap windows to preserve cross-boundary context. For tabular content, chunking mechanism 130 may emit row-, column-, and cell-level chunks alongside a synthesized natural-language summary. For figures and scanned regions, chunking mechanism 130 may associate OCR text and layout anchors to enable mixed text/layout retrieval.

In one or more embodiments, chunking mechanism 130 normalizes whitespace, punctuation, dates, and/or units of measure to UCUM. Chunking mechanism 130 may deduplicate near-identical spans via k-shingling and content hashes. Chunking mechanism 130 may apply adaptive policies to tune chunk size and overlap per corpus (e.g., tighter chunks for dense criteria sections and larger chunks for narrative guidelines). Chunking mechanism 130 may apply hierarchical chunking to maintain parent-child links across section/paragraph/sentence levels.

In one or more embodiments, prior authorization decision support engine 104 includes an embedding mechanism 132. Embedding mechanism 132 performs one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by ML algorithms.

In some embodiments, the vector embedding functions are word embedding techniques. Word embedding techniques use NLP and ML to represent words as dense vectors of real numbers. Word embedding techniques aim to capture the semantic and syntactic meaning of words as well as their relationships with other words in a language. Word embedding techniques include Term Frequency-Inverse Document Frequency (TF-IDF), Word2Vec, Global Vectors (GLOVE), Large Language Models (LLMs), BioWordVec fastText, and Bidirectional Encoder Representation (BERT).

In one or more embodiments, the word embedding techniques include Self-Alignment Pretraining for Biomedical Entity Representations (SAPBERT). The SAPBERT is a pre-trained BERT model that is trained in Medical Entity Linking (MEL) tasks. MEL maps various entities to unified concepts in the medical knowledge graph. Word representation learning faces a significant challenge due to the existence of heterogeneous names. For example, in healthcare, terms like ‘nostril’ and ‘nare’ are used interchangeably but yield considerably different embedding representations when generated by models not specifically trained for MEL. SAPBERT works on self-alignment of biomedical entity representation, so semantically similar entities belonging to the same concept are brought closer in the embedding space, forming compact clusters. SAPBERT leverages UMLS, the largest collection of biomedical concepts and synonyms, and collates the synonyms from various controlled vocabularies, e.g., SNOMED CT, MeSH, Gene Ontology, RxNorm, and OMIM. SAPBERT performs better compared to other variants of BERT like Bio-BERT and Clinical-BERT with respect to the MEL challenges. The SAPBERT model can accurately capture fine-grained semantic relationships and heterogeneous naming in the biomedical domain compared to other variants of BERT. The ability of SAPBERT to handle out-of-vocabulary terms, misspelled words, and rare medical terms provides a significant advantage over other models.

In one or more embodiments, embedding mechanism 132 hosts one or more encoder pipelines: (i) text encoders (e.g., transformer dual encoders) for narrative policy text, clinical notes, and plan documents; (ii) code-aware encoders that enrich text with standardized terminology features (ICD-10-CM/PCS, CPT/HCPCS, LOINC with UCUM units, RxNorm, SNOMED CT), including learned code embeddings and sparse one-hot or hash-bucket vectors; and/or (iii) structured-quantity encoders that serialize observations into canonical templates (e.g., “potassium 5.2 mmol/L on 2025 Aug. 1”) after unit normalization to UCUM and then embed them or append compact numeric features (value z-scores, trend slopes) to the text vector.

In one or more embodiments, embedding mechanism 132 performs preprocessing (language/charset detection, text normalization, punctuation/whitespace harmonization, PHI redaction where required, and de-duplication via content hashes) and/or tokenization with corpus-specific rules (e.g., preserving code tokens like “73721” or “I10” as single units). Embedding mechanism 132 may support multilingual corpora and domain adapters, mean/CLS pooling, and optional L2 normalization to enable cosine similarity.

In one or more embodiments, prior authorization decision support engine 104 includes a similarity score calculator 134. Similarity score calculator 134 refers to hardware and/or software configured to perform the operations described herein for computing, normalizing, and/or calibrating relevance scores between a query request vector embedding and candidate clinical guideline vector embeddings. Similarity score calculator 134 calculates a similarity between vector embeddings for a query request and candidate clinical guidelines. To measure similarity, a distance measure or similarity metric is chosen. Common distance measures include Euclidean distance, cosine similarity, and Jaccard similarity. When dealing with large data sets, an index may be created to speed up the search process. An index is a data structure that organizes the data in a way that allows for efficient retrieval of similar items.

In one or more embodiments, prior authorization decision support engine 104 includes a reranking mechanism 136. Reranking mechanism 136 refers to hardware and/or software configured to perform the operations described herein for reordering a candidate set of clinical guidelines. Reranking mechanism 136 may assist in maximizing decision-time relevance and policy compliance in the prior-authorization workflows. Reranking mechanism 136 may ingest a list of candidates (e.g., guideline fragments, plan rules, patient-evidence pointers) accompanied by calculator outputs—namely a unified similarity score in [0,1][0,1][0,1], per-feature contributions (dense/lexical/code overlaps, temporal/unit proximity), and candidate metadata (issuer, policy identifier/version/effective dates, product line and site-of-care applicability, clause type, section/paragraph anchors, chunk lineage, and provenance hashes).

In one or more embodiments, reranking mechanism 136 applies deterministic filters that zero or exclude items failing hard constraints (outside effective/sunset windows, product-line/site mismatch, issuer precedence violations). Reranking mechanism 136 may collapse near-duplicates using shingling/content hashes and version lineage (retaining the highest-utility representative). Reranking mechanism 136 may compose a rerank feature vector per candidate that includes the following: (i) the calculator's calibrated similarity score; (ii) policy-fit indicators (effective-date gap, product-line/site compatibility); (iii) clinical specificity features (presence of thresholds, temporal windows, step-therapy or contraindication gates); (iv) structural features (heading depth, table vs narrative, code-table coverage); (v) recency/version features; and/or (vi) diversity/conflict signals (coverage vs non-coverage pairings for the same code/indication). A learning-to-rank model (pointwise/pairwise/listwise) and/or a cross-encoder scorer (joint query-candidate transformer) may produce a refined relevance score. Reranking mechanism 136 may apply score fusion to combine a refined score with a calculator's score via learned or calibrated weights to yield a final confidence.

In one or more embodiments, reranking mechanism 136 enforces list-level constraints and diversification (e.g., Maximal Marginal Relevance). This may ensure that a top-K set covers complementary rationale types (coverage indications, documentation requirements, coding tables) and avoids redundant fragments from the same clause/version. Tie-breakers ensure determinism (priority: higher fused score→closer effective date→issuer precedence→section priority→stable chunk ID). Outputs include the following: a ranked bundle with per-item final scores and confidence intervals; explanations listing the dominant features that changed their order relative to the similarity score calculator; and resolved conflict sets (e.g., overlapping coverage/non-coverage clauses) with the applied precedence rationale; and anchors (page/section/offsets) suitable for citation in downstream rationales. Reranking mechanism 136 supports batch and streaming modes, batched cross-encoding, early-exit heuristics, and/or caching for frequent code/indication queries.

In one or more embodiments, reranking mechanism 136 employs rerank-english-v3.0 as the reranking model.

In one or more embodiments, prior authorization decision support engine 104 includes a selection mechanism 138. Selection mechanism 138 refers to hardware and/or software configured to perform the operations described herein for selecting relevant clinical guidelines from a plurality of clinical guidelines. Selection mechanism 138 may select, as relevant, clinical guidelines associated with clinical guideline vector embedding having similarity score with a query vector embedding above a threshold. Alternatively, selection mechanism 138 may select, as relevant, a top number of clinical guidelines associated with clinical guideline vector embeddings ranked by similarity score with the query vector.

In one or more embodiments, prior authorization decision support engine 104 includes a feedback mechanism 140. Feedback mechanism 140 refers to hardware and/or software configured to ingest, validate, persist, and operationalize feedback signals generated before, during, and after prior-authorization (PA) adjudication with the objective of correcting case-level outcomes and continuously improving guideline execution, extraction models, routing logic, and service levels. Feedback mechanism 140 may accept events from multiple sources, including utilization reviewer overrides and annotations, provider portal corrections and document uploads, peer-to-peer outcomes, appeal decisions, post-service claims adjudication results, contradiction/consistency checks, SLA breach alerts, and/or model telemetry (e.g., low-confidence extractions or ranking disagreements). Events may be normalized to a schema that includes one or more of the following: event identifier, event type and severity, scope (decision-level, criterion-level, extractor-level, or policy-level), subject linkages (request ID, guideline/policy ID and effective version, criterion/node IDs, health-plan configuration version, patient-fact IDs with code/value/unit and timestamps), actor and role, provenance (channel, document IDs, page/section or character-offset anchors, parser/model versions), payload (reason codes plus human-readable rationale, attachments or structured deltas), confidence, consent/sensitivity labels, and/or creation/receipt timestamps. Feedback mechanism 140 may ingest feedback via APIs, message bus connectors, and UI tooling. A schema registry may enforce conformance; idempotency keys may prevent duplicate application; and a quarantine path may isolate malformed or policy-restricted events.

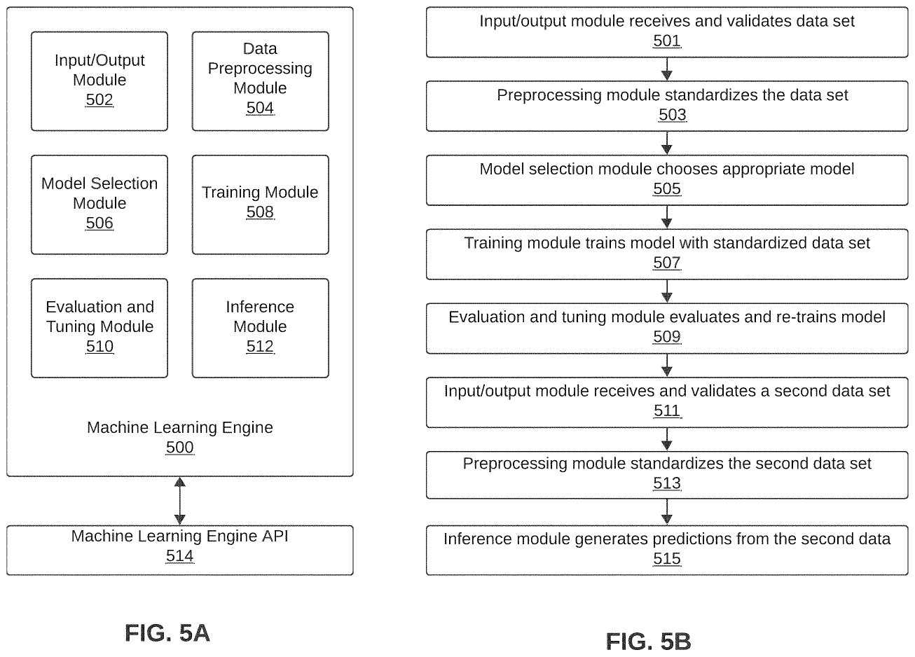

In one or more embodiments, prior authorization decision support engine 104 includes machine learning (ML) engine 142. ML engine 142 refers to hardware and/or software configured to perform the operations described herein for training and/or applying ML models. The structure and function of ML engine 142 will be described below in detail in Section 7, titled MACHINE LEARNING ARCHITECTURE and Section 8, titled MACHINE LEARNING ENGINE OPERATIONS, that reference FIGS. 5A and 5B, respectively.

In one or more embodiments, prior authorization decision support engine 104 includes generative AI model 144. Generative AI model 144 refers to hardware and/or software configured to perform the operations described herein for determining a prior authorization decision for medical procedures for patients. Generative AI model 144 may be a large language model (LLM), e.g., command-r-plus-08-2024—a refined LLM for reasoning tasks, and command-xlarge-nightly—a cutting edge LLM trained nightly for the latest updates and enhanced performance. Generative AI models are described in detail below in Section 9, titled GENERATIVE AI MODELS.

In one or more embodiments, interface 106 refers to hardware and/or software configured to facilitate communications between a user and prior authorization decision support engine 104. Interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of interface 106 are specified in different languages. The behavior of user interface elements is specified in a dynamic programming language such as JavaScript. The content of user interface elements is specified in a markup language, such as hypertext markup language (HTML) or XML User Interface Language (XUL). The layout of user interface elements is specified in a style sheet language such as Cascading Style Sheets (CSS). Alternatively, interface 106 is specified in one or more other languages, such as Java, C, or C++.

### 3. LEVERAGING GENERATIVE AI IN GENERATING PRIOR AUTHORIZATION REPORTS FOR MEDICAL PROCEDURES FOR PATIENTS

FIG. 2 illustrates an example set of operations for leveraging generative AI to generate prior authorization reports for medical procedures for patients in accordance with one or more embodiments. One or more operations illustrated in FIG. 2 may be modified, rearranged, or omitted. Accordingly, the particular sequence of operations illustrated in FIG. 2 should not be construed as limiting the scope of one or more embodiments.

One or more embodiments access a plurality of clinical guidelines and patient data, including health plan details, location of services, patient details, and request details (Operation 202). The system may connect to multiple sources,. e.g., payer medical-policy portals, society guidelines, internal policy repositories, and delegated UM vendors, via APIs, secure web retrieval, and/or file ingestion (PDF, HTML, DOCX, CSV).

In one or more embodiments, the system exposes unified read interfaces to health plan details, location of services, patient details, and/or request details. The system may source health plan details from eligibility/benefit transactions, plan masters, and/or formulary files. The system may normalized health detail plans to an interoperable representation (e.g., FHIR Coverage/InsurancePlan equivalents) with effective/sunset dates and provenance. The system may obtain patient details from heterogeneous sources, including EHR, claims, pharmacy, and imaging. The system may map patient details to standardized terminologies (SNOMED CT/ICD-10-CM for conditions; CPT/HCPCS for procedures; LOINC with UCUM units for labs; RxNorm for medications). The system may store request details as a versioned record for audit and exact replay.

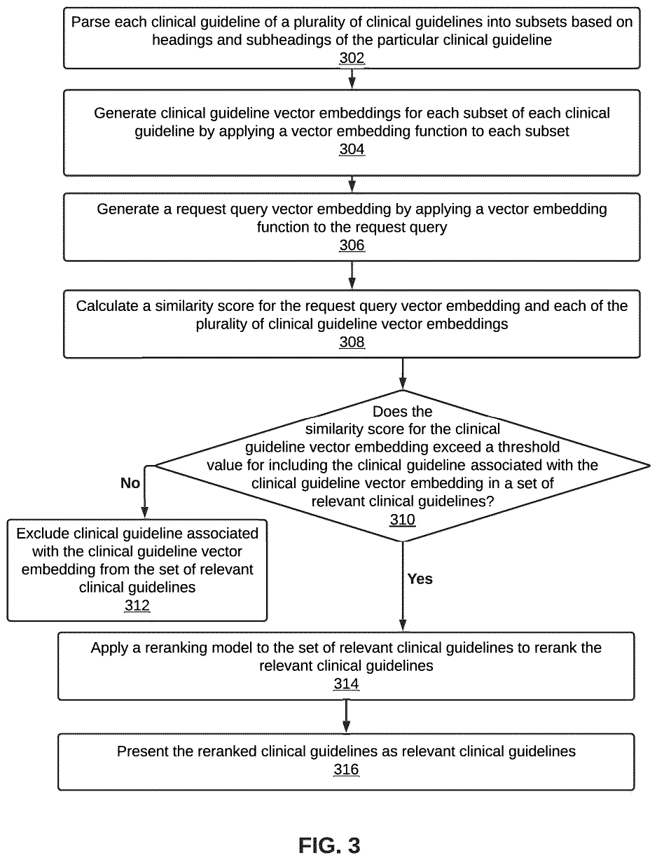

One or more embodiments determine relevant clinical guidelines for the prior authorization request from the plurality of clinical guidelines (Operation 204). Operations for determining relevant clinical guidelines form the plurality of clinical guidelines will be described below in section 4, titled determining relevant clinical guidelines for PRIOR AUTHORIZATION REQUESTS, that references FIG. 3.

One or more embodiments, generate a prompt for a generative AI model for generating a prior authorization report based on the patient data (Operation 206). The prompt may include a text input that the generative AI model uses to generate a response, i.e., authorization report. Components of a prompt generated by the system may include i) role assignment, ii) inputs, iii) output format, and iv) reasoning steps. Role assignment may include having the generative AI model respond as a medical expert evaluating requests for appropriateness and cost-effectiveness of a medical procedure. Inputs may include clinical notes, family history, diagnostic results, procedure requests, health plan details, CPT and ICD descriptions, supporting documents, and location of service details. Output format may include a decision, a table, recommendations, and a guideline source. Decisions may include approved, step therapy, alternative treatment, or denied. The Table summarizes symptoms, guidelines alignment, health plan coverage, and service location. Recommendation may include suggested alternatives. Guideline source may include citations to PDF references. Reasoning steps may include instructions to (a) evaluate clinical symptoms, family medical history, and diagnostic results, (b) match symptoms and request with guidelines, (c) assess an appropriateness of requested procedure for the location of service, and (d) verify compatibility with health plan coverage and alignment.

One or more embodiments, prepare the generative AI model to generate a prior authorization report by providing the prompt to the generative AI model (Operation 208). The system may normalize encodings, dates, and/or measurement units, validate codes, and deduplicate and/or minimize sensitive content according to consent policies using deterministic ranking and truncation rules that preserve high-utility evidence and mandatory policy text. The system may bind decoding parameters (temperature, top-p, max tokens, stop sequences) and generate a prompt fingerprint to ensure reproducibility. The system may transmit the prompt to a compliant inference endpoint over an encrypted channel. In streaming modes, the system may perform online validation to halt generation upon completion of the contract or detection of disallowed tokens. After generation, a validator may enforce the output contract by checking schema conformance, reference integrity, unit/code sanity, and logical consistency with the included rules.

One or more embodiments apply the generative AI model to the relevant clinical guideline and the patient data to determine a target clinical guideline and generate a prior authorization report in response to the prior authorization request (Operation 210). The information listed in the prompt as inputs, including clinical notes, family history, diagnostic results, procedure requests, health plan details, CPT and ICD descriptions, supporting documents, and location of service details, are provided to the generative AI model along with the target clinical guideline. The system may serialize this information as text and lightweight tables. The system may retain machine-resolvable identifiers and/or anchors (evidence IDs, policy clause IDs, timestamps, provenance, and effective dates), so the model can reference the identifiers and/or anchors deterministically. Prior to model invocation, the system may normalize dates/times, validate coding syntax (CPT/HCPCS, ICD-10-CM/PCS, LOINC, RxNorm, SNOMED CT), harmonize units to UCUM, deduplicate semantically equivalent facts, and filter content under consent and minimization policies to restrict exposure to the minimum necessary information.

One or more embodiments determine if the patient data is sufficient for responding to the prior authorization request (Operation 212). The system may determine that the patient data provided through the prior authorization interface is missing information. The system may employ a completeness and gap-analysis engine to determine missing information. The system may present a notification to the healthcare professional indicating additional information is required and requesting the additional information be provided. The system may automatically query a relevant database for the additional information.

One or more embodiments access missing patient data (Operation 214). The missing patient data may already exist but requires being located and uploaded to the system through the prior authorization interface. The missing patient data may include test results for tests that have not been completed, findings that have not been made, and/or treatments that have not been performed. In this manner, accessing the missing patient data necessitates running the tests, making the findings, and/or treating the patient. Once the tests have been completed, the findings have been made, and/or the treatment has been performed, the missing data may be provided to the system through the prior authorization request interface.

One or more embodiments generate a prior authorization report responsive to the prompt based on the target clinical guideline and the patient data (Operation 216). Using the format detailed in the prompt, the system generates the prior authorization report. The prior authorization report may include the following: (i) a decision responsive (e.g., approved, step therapy, alternative treatment, denied) to the medical procedure request; (ii) a table summarizing patient symptoms; (iii) alignment of the symptoms with the target clinical guideline; (iv) health plan coverage; (v) service location; (vi) recommendations; and/or (vii) a source for the target clinical guideline.

One or more embodiments present the prior authorization report in a user interface (Operation 218). The prior authorization report may include graphs, icons, and other visual elements for displaying the prior authorization information in the prior authorization request interface. A prior authorization report denying a request for authorization may include an interface element for appealing the decision. The system may automatically generate an appeal request in anticipation of denial by the insurer when a request is filed. In the event the denial was based on missing information, the prior authorization request interface may include a field for submitting additional information.

In one or more embodiments, when the system determines that a prior authorization request for a medical procedure for a patient is approved, the system initiates preparation for the medical procedure. The system may automatically initiate preparations for the medical procedure. Initiating preparations may require healthcare provider approval. Approval may be provided by interacting with an interface element in the prior authorization request interface. Initiating preparation for the medical procedure may include scheduling the procedure, updating the treatment plan, and notifying relevant parties. Scheduling the procedure may include arranging an operating room for the procedure, pre-and post-care accommodations for the patient, scheduling personnel, and ordering supplies. Updating the treatment plan of the patient may include amending the EHR of the patient to increase, decrease, initiate, or discontinue one or more medications in preparation for the procedure. Similarly, updating the treatment plan may include changes to the diet, activities, and/or routines of the patient in preparation for the procedure.

### 4. DETERMINING RELEVANT CLINICAL GUIDELINES FOR PRIOR AUTHORIZATION REQUESTS

FIG. 3 illustrates an example set of operations for determining target clinical guidelines for medical procedure requests for a patient in accordance with one or more embodiments. One or more operations illustrated in FIG. 3 may be modified, rearranged, or omitted. Accordingly, the particular sequence of operations illustrated in FIG. 3 should not be construed as limiting the scope of one or more embodiments.

One or more embodiments parse each clinical guideline of a plurality of clinical guidelines into subsets based on headings and subheadings of the particular clinical guideline (Operation 302). Parsing tools may be used on each of the clinical guidelines to identify the headings and the subheadings associated with the respective clinical guidelines to extract relevant text. The extracted text may be further segmented into smaller, contextually coherent chunks or subsets based on the headings and the subheadings of the clinical guidelines.

In one or more embodiments, the headings/subheadings of the clinical guidelines include “Description of Procedure or Service,” “Policy,” “Benefits Application,” “Policy Guidelines,” “Scientific Background and Reference Sources,” and “Policy Implementation/Update Information.” Segmentation of the clinical guidelines ensures that the extracted information remains aligned with an original guideline structure. Segmentation may also facilitate easier handling, organizing, and/or analyzing the clinical guidelines. Each subset may be ascribed various attributes, including source, page, heading, and content. Source is the file path for a clinical guideline associated with the subset/chunk. Page is the page number where the subset/chunk originated. Heading is the section heading of the subset/chunk and provides context for the subset/chunk. Content is the associated text content of the subset/chunk and captures detailed information.

In one or more embodiments, python-based parsing tools, e.g., pdfplumber library, are used to parse the pdfs of the clinical guidelines.

One or more embodiments generate clinical guideline vector embeddings for each subset of each clinical guideline by applying a vector embedding function to each subset (Operation 304). More particularly, the system applies a vector embedding function to the text of each subset or chunk associated with each clinical guideline of the plurality of clinical guidelines to generate vector embeddings for each subset. The vector embeddings may be indexed in a vector store.

In one or more embodiments, the text and contextual metadata for each subset/chunk are transformed into a high-dimensional vector embedding using Cohere's language model, e.g., embed-english-v3.0. Doman-specific language models (e.g., SapBERT, Bio-Clinical BERT) may be used to generate the vector embeddings. The vector embeddings for the headings and the vector embeddings for the content may be generated separately and combined using a weighted average. For example, a weight of 0.15 may be applied to the heading of the subset/chunk, and a weight of 0.85 may be applied to the content of the subset/chunk.

In one or more embodiments, text of each subset/chunk associated with each of the plurality of clinical guidelines is indexed into a vector store/vector database using Hierarchical Navigable Small World (HNSW). Alternatively, other indexers, such as inverted file index (IVF) can be utilized. Indexing the clinical guidelines in this manner enables efficient handling of the high-dimensional data. Domain-specific language models and general-purpose language models may be utilized to encode the semantic meaning of the text. This format is optimized for rapid, similarity-based retrieval in a retrieval-augmented generation (RAG) framework. A vector store, including the vector embeddings and associated metadata, may be saved to disk to ensure easier reload during queries.

One or more embodiments generate a request query vector embedding by applying a vector embedding function to the request query (Operation 306). Initially, the system creates a request query. The request query may include various attributes, e.g., procedure request details, a CPT description associated with the procedure request, an ICD description associated with the procedure request, and/or location of service details. The text of the attributes of the request query may be aggregated. The system may apply a vector embedding function to the text of each attribute of the request query to generate vector embeddings for the attributes and combine the vector embeddings for the attributes to form the request query vector embedding. Alternatively, the system may apply the vector embedding function to the aggregated text for the request query to generate a request query vector embedding. The vector embedding function may be the same as or different from the vector embedding function applied to the subsets/chunks of the clinical guidelines. Generating a vector embedding from the aggregated text for the request query captures the semantic meaning of the request query.

One or more embodiments calculate a similarity score for the request query vector embedding and each of the plurality of clinical guideline vector embeddings (Operation 308).

In one or more embodiments, the request query vector embedding is used to perform a k-nearest neighbor (k-NN) search against the plurality of clinical guideline vector embeddings stored in the HNSW index. Both the plurality of clinical guideline vector embeddings and the request query vector embedding are loaded into memory. The HNSW index is an optimized data structure that enables fast similarity search. The clinical guidelines file may be used for reference to fetch the text associated with the clinical guideline vector embeddings identified as relevant. The k-NN search identifies a top k clinical guideline vector embeddings and associated clinical guidelines.

In one or more embodiments, top k clinical guideline vector embeddings are filtered. Filtering the top k clinical guideline vector embeddings may include determining a similarity score for each of the top k clinical guideline vector embeddings and the request query vector embedding. The similarity score may be calculated using cosine similarity.

One or more embodiments determine that the similarity score for the clinical guideline vector embedding exceeds a threshold value for including the clinical guideline associated with the clinical guideline vector embedding as a set of relevant clinical guidelines (Operation 310). Similarity scores may range from −1.0 to 1.0. The closer to 1.0 the similarity score, the greater the similarity between the clinical guideline vector embedding and the request query vector embedding. When vector embeddings are non-negative, cosine similarity is between 0 and 1. A user may determine a threshold value for the similarity. In an example, the threshold value is 0.5. In this manner, each clinical guideline that is associated with a clinical guideline vector embedding in the top k clinical guideline vector embeddings and that has a similarity score of 0.5 or higher with the request query vector embedding is deemed relevant.

One or more embodiments exclude clinical guideline associated with the clinical guideline vector embedding from the set of relevant clinical guidelines (Operation 312). The filter may exclude top k clinical guidelines vector embeddings with a similarity score under a threshold score, e.g., 0.5. In this manner, each clinical guideline that is associated with a clinical guideline vector embedding in the top k clinical guideline vector embeddings and that has a similarity score below 0.5 with the request query vector embedding is filtered from the set of retrieved clinical guidelines to create a set of relevant clinical guidelines.

One or more embodiments apply a reranking model to the set of relevant clinical guidelines to rerank the set of relevant clinical guidelines (Operation 314). More particularly, the system applies the reranking model to the set of relevant clinical guidelines, i.e., the clinical guideline vector embeddings for the top k clinical guideline vector embeddings that have a similarity score that exceeds the threshold. The reranking model may evaluate the clinical guidelines and associated clinical guideline vector embeddings using multiple fields, such as guideline, criteria, and/or details, to ensure that best matches are reranked highest.

In one or more embodiments, a reranking model refines and reorders the relevant clinical vector embeddings based on relevance by evaluating the semantic relationship between the results and the query. The reranking model may ingest the top-K candidates and construct, for each (q, di)(q, d_i)(q, di) pair, an augmented feature vector that includes (i) a cross-encoder or late-interaction score produced by jointly encoding the query and candidate text so token-level alignments are directly compared; (ii) code-aware overlaps for standardized terminologies (ICD-10-CM, CPT/HCPCS, LOINC/UCUM, RxNorm, SNOMED CT) including hierarchical proximity and unit-normalized numeric matches; (iii) temporal fit features that verify look-back windows, frequency limits, and effective dates; and (iv) structure/policy-fit indicators (section/heading type, clause class, site-of-care or product-line compatibility). A learned scoring function (pointwise or listwise learning-to-rank) or a cross-encoder head may produce calibrated relevance scores. The reranking model may down-weight or remove candidates failing hard constraints (e.g., outside effective window, wrong site of care). The reranking model may emit a deterministic ordering after optional diversification (e.g., Maximal Marginal Relevance) to balance coverage of rationale types. The system may use positive pairs and/or hard negatives mined from initial retrieval to train the reranking model to elevate semantically correct, policy-conforming clauses while demoting near-misses, thereby improving top-K precision without re-embedding the corpus.

One or more embodiments present the reranked clinical guidelines as relevant clinical guidelines (Operation 316). The system may automatically provide the clinical guidelines associated with the top reranked clinical guideline vector embeddings as the relevant clinical guidelines. The system may automatically identify the clinical guideline associated with the top clinical guideline vector embedding of the reranked clinical guideline vector embeddings as the most relevant clinical guideline or the target clinical guideline. The system may then provide the relevant clinical guidelines and/or the target clinical guideline to a generative AI model for generating the prior authorization report for the requested procedure.

### 5. EXAMPLE PRIOR AUTHORIZATION REQUEST INTERFACE AND PRIOR AUTHORIZATION REPORT

A detailed example is described below for purposes of clarity. Components and/or operations described below should be understood as one specific example which may not be applicable to certain embodiments. Accordingly, components and/or operations described below should not be construed as limiting the scope of any of the claims.

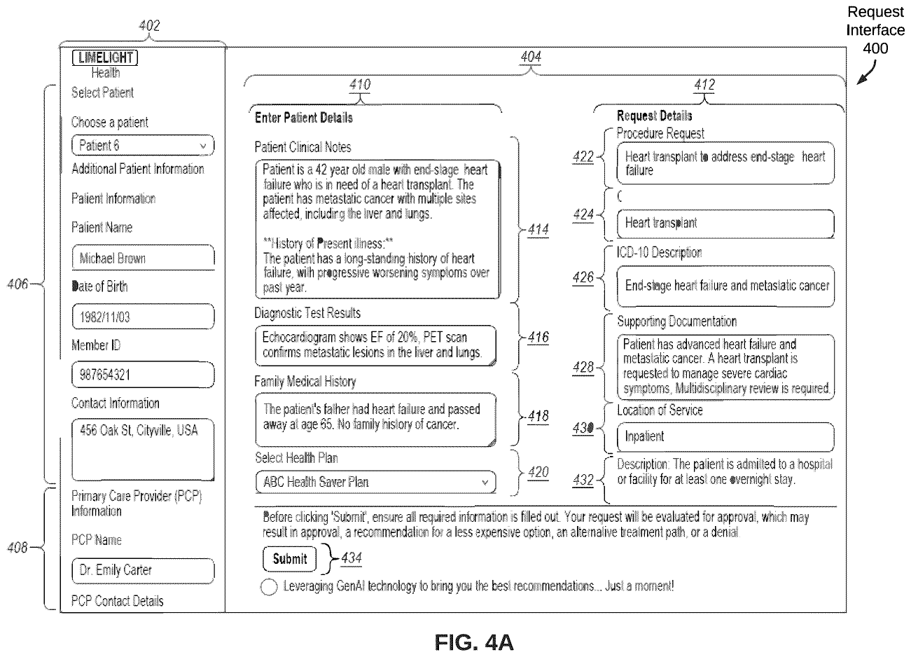

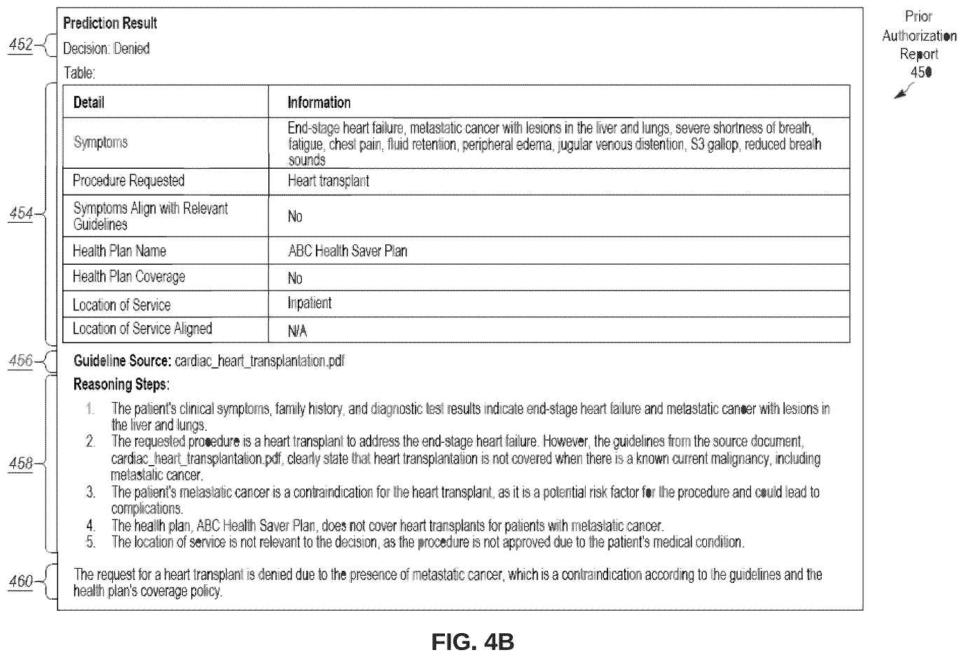

FIG. 4A illustrates an example prior authorization request interface 400 of a prior authorization decision support interface for requesting prior authorization for a medical procedure for a patient. As shown, prior authorization request interface 400 includes an identification interface 402 for identifying a patient, a primary care provider (PCP), and a details interface 404 for providing patient details and request details.

Identification interface 402 includes patient fields 406 for entering patient data, including patient details and request details, as well as PCP fields 408. Patient fields 406 include a pull-down menu to choose a patient, and fields for additional patient information. As shown, patient information includes patient name, date of birth, member ID, and contact information. PCP fields may include PCP name and PCP contact details. This information may be provided as text boxes. The text boxes may be filled in manually or auto populated by the system.

Details interface 404 includes patient detail fields 410 and request detail fields 412. As shown, patient detail fields 410 include patient clinical notes 414, diagnostic test results notes 416, family medical history 418, and health plan 420. Request detail fields 412 include procedure request 422, CPT description 424, ICD-10 description 426, supporting documentation 428, location of service 430, and/or description 432. Patient detail fields 410 and/or request detail fields 412 may be filled in manually or auto populated by the system.

Details interface 404 may further include an interface button 434 for submitting a prior authorization request. Interface button 434 may be manually selected by a healthcare professional after filling in patient detail fields 410 and request detail fields 412.

FIG. 4B illustrates an example prior authorization report 450 generated in response to a prior authorization request. Prior authorization report 450 includes a decision 452, a table 454 detailing the request, a relevant guideline source 456, reasoning steps 458, and an explanation 460. As shown, decision 452 indicates that the prior authorization request was “Denied.” Table 454 includes request details, such as symptoms, procedure requested, indication of alignment with symptoms, health plan name, health plan coverage, location of service, and/or indication of alignment with location of service. Relevant guideline source 456 includes a source for the guideline applied in the prior authorization request. Reasoning steps 458 detail the reasoning for the prior authorization decision. Explanation 460 is a concise summary of the information used in determining the decision.

### 6. PRACTICAL APPLICATIONS, ADVANTAGES & IMPROVEMENTS

Leveraging generative AI to generate prior authorization reports for medical procedures for patients provides various practical applications. Practical applications include automated intake and triage, guideline compilation and maintenance from payer policy text, RAG-guided decision drafting with per-criterion citations, targeted evidence requests that enumerate the smallest additional documentation required, generation of member/provider communications (initial, pend, denial, overturn letters with jurisdictional disclosures), support for appeals and peer-to-peer reviews through case summaries and talking points, real-time point-of-care determinations for straightforward cases, and quality/compliance reporting with SLA timelines and clause-level heatmaps.

The capabilities provided by the prior authorization decision support system yield numerous practical outcomes, such as increased automation of routine cases, reductions in median turnaround time for initial determinations, fewer and more precise pend cycles, lower appeal sustain rates due to consistent clause-based reasoning, and/or higher reviewer throughput on complex queues. Security and governance controls, including tenant isolation, encryption in transit/at rest, consent/sensitivity labeling, role-based access, and/or change management, ensure that the generative AI-enabled prior authorization process remains reproducible, privacy-preserving, and/or compliant while delivering faster, more consistent, and more transparent authorization decisions.

Implementing generative AI in prior authorization decision making provides many advantages, including scalability and efficiency, enhanced accuracy and consistency, reduced administrative burden, better audit trails and transparency, and improved patient experience. The prior authorization decision support system delivers measurable advantages in speed, consistency, and auditability. By extracting clinically material facts from unstructured documents and aligning them to standardized terminologies (ICD-10-CM/PCS, CPT/HCPCS, LOINC with UCUM units, RxNorm, SNOMED CT), generative AI reduces false pends and denials arising from “missing” evidence that is present but poorly surfaced. Schema-constrained generation and post-hoc validation may yield deterministic, machine-readable outputs suitable for straight-through processing. Explicit citations to policy clause anchors and evidence identifiers improve explainability and reduce variance across reviewers. Policy agility may be enhanced through automated parsing and versioning of updated payer guidance, enabling timely adherence to effective-date changes. Administrative burden may be lowered by automatically drafting rationale, documentation checklists, and correspondence, allowing clinical reviewers to focus on edge cases and complex judgment rather than document retrieval and formatting.

The presently disclosed prior authorization decision support system provides many improvements over existing systems, including enhanced document understanding, real-time adaptation to evolving guidelines, improved accuracy and consistency, proactive communication and explanation, greater stability and efficiency, automated learning from past decisions, reduced appeal and rework cycle, and improved user experience and satisfaction.

More particularly, improvements over existing systems include the following: (i) RAG with policy overlays, which merges payer-and product-specific clauses (e.g., site-of-care restrictions, step-therapy bypasses) onto base guideline text and requires the model to ground determinations in cited clauses; (ii) counterfactual reasoning, which computes and presents the minimal evidence set needed to satisfy unmet criteria instead of issuing broad, generic pend requests; (iii) confidence-aware, clause-level scoring, which propagates source reliability and recency to criterion outcomes and escalates low-confidence or contradictory cases to human review; (iv) schema-locked, validator-backed outputs with automated repair prompts that enforce format, reference integrity, unit/code sanity, and logical consistency with governing rules; (v) continuous learning loops that incorporate reviewer overrides, appeal outcomes, and claims adjudication signals to recalibrate extractors, thresholds, and templates under strict version control; (vi) fairness and drift monitoring, that tracks approval/pending/denial distributions and reversal rates across cohorts (without storing protected attributes) and triggers governance when behavior shifts after policy or model updates; and (vii) token-budget optimization and PHI minimization, which select high-utility facts within model context limits while redacting or truncating sensitive data per consent and “minimum necessary” policies. End-to-end version pinning binds each determination to exact policy versions, plan configuration, prompts, model parameters, and data snapshots, enabling exact replay for audit or appeal and strengthening regulatory compliance.

The generative AI techniques described herein further mitigate the need to deploy software patches every time there are changes to prior authorization rules or new rules are accessed. The system may dynamically ingest rules and unstructured clinical inputs without the need to execute computationally expensive or disruptive tasks, such as patch updates or model retraining. Updates may further be ingested without requiring a developer or other professional to program in the changes or otherwise have knowledge of the underlying system architecture. The system may thus avoid instability associated with hard-coded rule engines while improving inference accuracy using the vector embedding techniques previously described. As a result, overall system performance and reliability may be improved.

The system further provides for asynchronous workflow integration, which may improve computing efficiency. For example, an automated workflow module within an EHR may poll a database repeatedly to check for updates, such as to determine whether a provider response has arrived or whether all clinical fields are complete. The polling model typically generates large volumes of redundant queries against an EHR database. The increased database transaction load may slow query execution for other processes due to query contention and locking, which may further cause UI latency when clinicians or other users are interacting with the computing application. The generative AI-based processes above do not rely on polling. The processes may be driven by asynchronous event triggers, thereby reducing wasted calls to the database incurred by polling-based workflow integrations. By reducing database load, fewer CPU and I/O cycles are spent handling unnecessary queries, which lowers latency and improves overall system throughput.

### 7. MACHINE LEARNING ARCHITECTURE

FIG. 5A illustrates a machine learning engine 500 in accordance with one or more embodiments. As illustrated in FIG. 5A, machine learning engine 500 includes input/output module 502, data preprocessing module 504, model selection module 506, training module 508, evaluation and tuning module 510, and inference module 512.

In accordance with an embodiment, input/output module 502 serves as the primary interface for data entering and exiting the system, managing the flow and integrity of data. This module may accommodate a wide range of data sources and formats to facilitate integration and communication within the machine learning architecture.

In an embodiment, an input handler within input/output module 502 includes a data ingestion framework capable of interfacing with various data sources, such as databases, APIs, file systems, and real-time data streams. This framework is equipped with functionalities to handle different data formats (e.g., CSV, JSON, XML) and efficiently manage large volumes of data. It includes mechanisms for batch and real-time data processing that enable the input/output module 502 to be versatile in different operational contexts, whether processing historical datasets or streaming data.

In accordance with an embodiment, input/output module 502 manages data integrity and quality as it enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the machine learning process.

In an embodiment, an output handler within input/output module 502 includes an output framework designed to handle the distribution and exportation of outputs, predictions, or insights. Using the output framework, input/output module 502 formats these outputs into user-friendly and accessible formats, such as reports, visualizations, or data files compatible with other systems. Input/output module 502 also ensures secure and efficient transmission of these outputs to end-users or other systems in an embodiment and may employ encryption and secure data transfer protocols to maintain data confidentiality.

In accordance with an embodiment, data preprocessing module 504 transforms data into a format suitable for use by other modules in machine learning engine 500. For example, data preprocessing module 504 may transform raw data into a normalized or standardized format suitable for training ML models and for processing new data inputs for inference. In an embodiment, data preprocessing module 504 acts as a bridge between the raw data sources and the analytical capabilities of machine learning engine 500.

In an embodiment, data preprocessing module 504 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods like imputation or removal of incomplete records, depending on the nature and volume of the missing data. Data preprocessing module 504 may be configured to handle anomalies in different ways depending on context. Data preprocessing module 504 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model's ability to learn effectively.

In an embodiment, data preprocessing module 504 includes a feature encoding framework that ensures categorical variables are transformed into a format that can be easily interpreted by machine learning algorithms. Techniques like one-hot encoding or label encoding may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 504 processes new data for inference, data preprocessing module 504 replicates the same preprocessing steps to ensure consistency with the training data format. This helps to avoid discrepancies between the training data format and the inference data format, thereby reducing the likelihood of inaccurate or invalid model predictions.

In an embodiment, model selection module 506 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 506 employs a variety of statistical and analytical techniques to understand data patterns, identify potential correlations, and assess the complexity of the task. Based on this analysis, it then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 506 utilizes techniques from the field of Automated Machine Learning (AutoML). AutoML systems automate the process of model selection by rapidly prototyping and evaluating multiple models. They use techniques like Bayesian optimization, genetic algorithms, or reinforcement learning to explore the model space efficiently. Model selection module 506 may use these techniques to evaluate each candidate model based on performance metrics relevant to the task. For example, accuracy, precision, recall, or F1 score may be used for classification tasks and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The mean squared error (MSE) metric may be used for regression tasks. MSE measures the average squared difference between the actual and predicted values, providing an indication of the model's accuracy. A lower MSE may indicate a model's greater accuracy in predicting values, as it represents a smaller average discrepancy between the actual and predicted values.

In accordance with an embodiment, model selection module 506 also considers computational efficiency and resource constraints. This is meant to help ensure the selected model is both accurate and practical in terms of computational and time requirements. In an embodiment, certain features of model selection module 506 are configurable such as a configured bias toward (or against) computational efficiency.

In accordance with an embodiment, training module 508 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly. Training module 508 handles the iterative process of feeding the training data into the model, adjusting the model's internal parameters (like weights in neural networks) through backpropagation and optimization algorithms, such as stochastic gradient descent or other algorithms providing similarly useful results.

In accordance with an embodiment, training module 508 manages overfitting, where a model learns the training data too well, including its noise and outliers, at the expense of its ability to generalize to new data. Techniques such as regularization, dropout (in neural networks), and early stopping are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 508 includes logic to handle different types of data and learning tasks. For instance, it includes different training routines for supervised learning (where the training data comes with labels) and unsupervised learning (without labeled data). In the case of deep learning models, training module 508 also manages the complexities of training neural networks that include initializing network weights, choosing activation functions, and setting up neural network layers.

In an embodiment, evaluation and tuning module 510 incorporates dynamic feedback mechanisms and facilitates continuous model evolution to help ensure the system's relevance and accuracy as the data landscape changes. Evaluation and tuning module 510 conducts a detailed evaluation of a model's performance. This process involves using statistical methods and a variety of performance metrics to analyze the model's predictions against a validation dataset. The validation dataset, distinct from the training set, is instrumental in assessing the model's predictive accuracy and its capacity to generalize beyond the training data. The module's algorithms meticulously dissect the model's output, uncovering biases, variances, and the overall effectiveness of the model in capturing the underlying patterns of the data.

In an embodiment, evaluation and tuning module 510 performs continuous model tuning by using hyperparameter optimization. Evaluation and tuning module 510 performs an exploration of the hyperparameter space using algorithms, such as grid search, random search, or more sophisticated methods like Bayesian optimization. Evaluation and tuning module 510 uses these algorithms to iteratively adjust and refine the model's hyperparameters - settings that govern the model's learning process but are not directly learned from the data - to enhance the model's performance. This tuning process helps to balance the model's complexity with its ability to generalize and attempts to avoid the pitfalls of underfitting or overfitting.

In an embodiment, evaluation and tuning module 510 integrates data feedback and updates the model. Evaluation and tuning module 510 actively collects feedback from the model's real-world applications, an indicator of the model's performance in practical scenarios. Such feedback can come from various sources depending on the nature of the application. For example, in a user-centric application like a recommendation system, feedback might comprise user interactions, preferences, and responses. In other contexts, such as predicting events, it might involve analyzing the model's prediction errors, misclassifications, or other performance metrics in live environments.

In an embodiment, feedback integration logic within evaluation and tuning module 510 integrates this feedback using a process of assimilating new data patterns, user interactions, and error trends into the system's knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and tuning module 510 employs version control mechanisms to track changes, modifications, and the evolution of the model, facilitating transparency and allowing for rollback if necessary. This continuous learning and adaptation cycle, driven by real-world data and feedback, helps to endure the model's ongoing effectiveness, relevance, and accuracy.

In an embodiment, inference module 512 transforms data raw data into actionable, precise, and contextually relevant predictions. In addition to processing and applying a trained model to new data, inference module 512 may also include post-processing logic that refines the raw outputs of the model into meaningful insights.

In an embodiment, inference module 512 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 512 transforms the outputs of a trained model into definitive classifications. Inference module 512 employs the underlying model as a tool to generate probabilistic outputs for each potential class. It then engages in an interpretative process to convert these probabilities into concrete class labels.

In an embodiment, when inference module 512 receives the probabilistic outputs from the model, it analyzes these probabilities to determine how they are distributed across some or every potential class. If the highest probability is not significantly greater than the others, inference module 512 may determine that there is ambiguity or interpret this as a lack of confidence displayed by the model.

In an embodiment, inference module 512 uses thresholding techniques for applications where making a definitive decision based on the highest probability might not suffice due to the critical nature of the decision. In such cases, inference module 512 assesses if the highest probability surpasses a certain confidence threshold that is predetermined based on the specific requirements of the application. If the probabilities do not meet this threshold, inference module 512 may flag the result as uncertain or defer the decision to a human expert. Inference module 512 dynamically adjusts the decision thresholds based on the sensitivity and specificity requirements of the application, subject to calibration for balancing the trade-offs between false positives and false negatives.

In accordance with an embodiment, inference module 512 contextualizes the probability distribution against the backdrop of the specific application. This involves a comparative analysis, especially in instances where multiple classes have similar probability scores, to deduce the most plausible classification. In an embodiment, inference module 512 may incorporate additional decision-making rules or contextual information to guide this analysis, ensuring that the classification aligns with the practical and contextual nuances of the application.

In regression models, where the outputs are continuous values, inference module 512 may engage in a detailed scaling process in an embodiment. Outputs, often normalized or standardized during training for optimal model performance, are rescaled back to their original range. This rescaling involves recalibration of the output values using the original data's statistical parameters, such as mean and standard deviation, ensuring that the predictions are meaningful and comparable to the real-world scales they represent.

In an embodiment, inference module 512 incorporates domain-specific adjustments into its post-processing routine. This involves tailoring the model's output to align with specific industry knowledge or contextual information. For example, in financial forecasting, inference module 512 may adjust predictions based on current market trends, economic indicators, or recent significant events, ensuring that the outputs are both statistically accurate and practically relevant.

In an embodiment, inference module 512 includes logic to handle uncertainty and ambiguity in the model's predictions. In cases where inference module 512 outputs a measure of uncertainty, such as in Bayesian inference models, inference module 512 interprets these uncertainty measures by converting probabilistic distributions or confidence intervals into a format that can be easily understood and acted upon. This provides users with both a prediction and an insight into the confidence level of that prediction. In an embodiment, inference module 512 includes mechanisms for involving human oversight or integrating the instance into a feedback loop for subsequent analysis and model refinement.

In an embodiment, inference module 512 formats the final predictions for end-user consumption. Predictions are converted into visualizations, user-friendly reports, or interactive interfaces. In some systems, like recommendation engines, inference module 512 also integrates feedback mechanisms, where user responses to the predictions are used to continually refine and improve the model, creating a dynamic, self-improving system.

### 8. MACHINE LEARNING ENGINE OPERATIONS

FIG. 5B illustrates the operation of a machine learning engine in one or more embodiments. In an embodiment, input/output module 502 receives a dataset intended for training (Operation 501). This data can originate from diverse sources, like databases or real-time data streams, and in varied formats, such as CSV, JSON, or XML. Input/output module 502 assesses and validates the data, ensuring its integrity by checking for consistency, data ranges, and types.

In an embodiment, training data is passed to data preprocessing module 504. Here, the data undergoes a series of transformations to standardize and clean it, making it suitable for training ML models (Operation 503). This involves normalizing numerical data, encoding categorical variables, and handling missing values through techniques like imputation.

In an embodiment, prepared data from the data preprocessing module 504 is then fed into model selection module 506 (Operation 505). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 508 trains the selected model with the prepared dataset (Operation 507). It implements learning algorithms to adjust the model's internal parameters, optimizing them to identify patterns and relationships in the training data. Training module 508 also addresses the challenge of overfitting by implementing techniques, like regularization and early stopping, ensuring the model's generalizability.

In an embodiment, evaluation and tuning module 510 evaluates the trained model's performance using the validation dataset (Operation 509). Evaluation and tuning module 510 applies various metrics to assess predictive accuracy and generalization capabilities. It then tunes the model by adjusting hyperparameters, and if needed, incorporates feedback from the model's initial deployments, retraining the model with new data patterns identified from the feedback.

In an embodiment, input/output module 502 receives a dataset intended for inference. Input/output module 502 assesses and validates the data (Operation 511).

In an embodiment, data preprocessing module 504 receives the validated dataset intended for inference (Operation 513). Data preprocessing module 504 ensures that the data format used in training is replicated for the new inference data, maintaining consistency and accuracy for the model's predictions.

In an embodiment, inference module 512 processes the new data set intended for inference, using the trained and tuned model (Operation 515). It applies the model to this data, generating raw probabilistic outputs for predictions. Inference module 512 then executes a series of post-processing steps on these outputs, such as converting probabilities to class labels in classification tasks or rescaling values in regression tasks. It contextualizes the outputs as per the application's requirements, handling any uncertainty in predictions and formatting the final outputs for end-user consumption or integration into larger systems.

In an embodiment, machine learning engine API 514 allows for applications to leverage machine learning engine 500. In an embodiment, machine learning engine API 514 may be built on a RESTful architecture and offer stateless interactions over standard HTTP/HTTPS protocols. Machine learning engine API 514 may feature a variety of endpoints, each tailored to a specific function within machine learning engine 500. In an embodiment, endpoints such as /submitData facilitate the submission of new data for processing, while /retrieveResults is designed for fetching the outcomes of data analysis or model predictions. The MLE API may also include endpoints like /pdateModel for model modifications and /trainModel to initiate training with new datasets.

In an embodiment, machine learning engine API 514 is equipped to support SOAP-based interactions. This extension involves defining a WSDL (Web Services Description Language) document that outlines the API's operations and the structure of request and response messages. In an embodiment, machine learning engine API 514 supports various data formats and communication styles. In an embodiment, machine learning engine API 514 endpoints may handle requests in JSON format or any other suitable format. For example, machine learning engine API 514 may process XML, and it may also be engineered to handle more compact and efficient data formats, such as Protocol Buffers or Avro, for use in bandwidth-limited scenarios.

In an embodiment, machine learning engine API 514 is designed to integrate WebSocket technology for applications necessitating real-time data processing and immediate feedback. This integration enables a continuous, bi-directional communication channel for a dynamic and interactive data exchange between the application and machine learning engine 500.

### 9. GENERATIVE AI MODELS

A generative AI model is a machine learning model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a large language model. Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a softmax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 502, when used for large language models, handles textual data, converting input text into a format that the model can process. This typically involves tokenization, where the text is broken down into manageable pieces, such as words or subwords, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 504 in the context of large language models may include steps such as normalization, where the text is converted to a uniform case and punctuation is standardized. This process ensures that the model treats similar words or symbols consistently, reducing the complexity of the input space. Additionally, techniques such as sentence segmentation may be applied to manage longer texts, enabling the model to process information in chunks that align with natural language structures.

In accordance with one or more embodiments, model selection module 506, when used for large language models involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 508, when used for large language models, is configured to adjust the model's parameters through exposure to training data. This process utilizes optimization algorithms, such as stochastic gradient descent, to minimize the difference between the model's predictions and the actual desired outputs. The training process is computationally intensive, often requiring specialized hardware such as GPUs (Graphics Processing Units) or TPUs (Tensor Processing Units) to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model's performance on new data).

In accordance with one or more embodiments, evaluation and tuning module 510 assesses the performance of large language models using metrics such as perplexity, accuracy, and F1 score, depending on the specific language tasks. Evaluation may involve comparing the model's output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 512, in the context of large language models, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

The self-attention mechanism, which is part of a transformer network, enables the model to weigh the importance of different elements within an input sequence, regardless of their position. This allows the model to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### 10. HARDWARE OVERVIEW

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

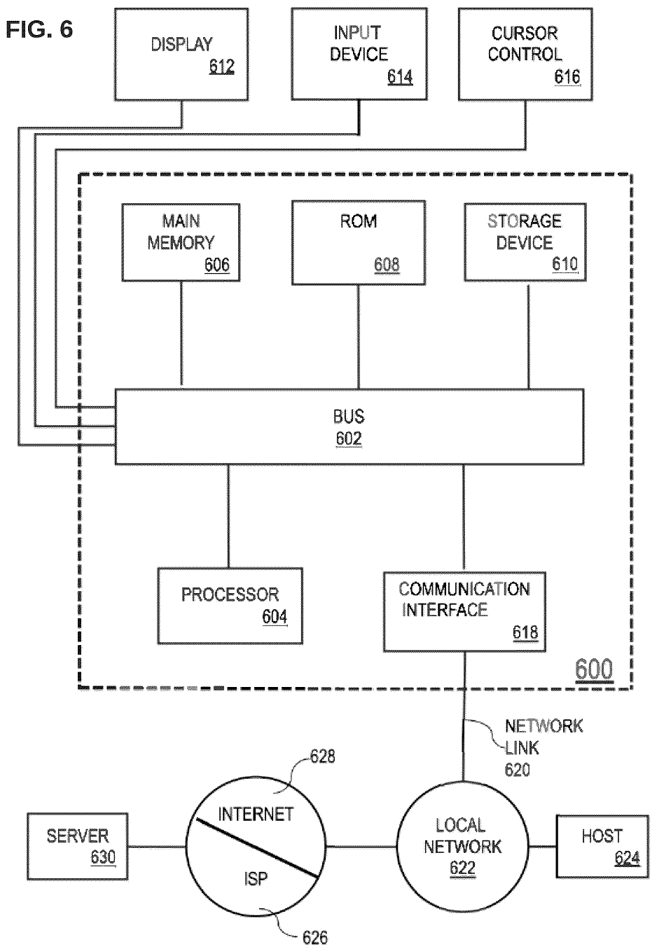

For example, FIG. 6 is a block diagram that illustrates a computer system 600 upon which an embodiment of the disclosure may be implemented. Computer system 600 includes a bus 602 or other communication mechanism for communicating information, and a hardware processor 604 coupled with bus 602 for processing information. Hardware processor 604 may be, for example, a general purpose microprocessor.

Computer system 600 also includes a main memory 606, such as a random access memory (RAM) or other dynamic storage device, coupled to bus 602 for storing information and instructions to be executed by processor 604. Main memory 606 also may be used for storing temporary variables or other intermediate information during execution of instructions to be executed by processor 604. Such instructions, when stored in non-transitory storage media accessible to processor 604, render computer system 600 into a special-purpose machine that is customized to perform the operations specified in the instructions.

Computer system 600 further includes a read-only memory (ROM) 608 or other static storage device coupled to bus 602 for storing static information and instructions for processor 604. A storage device 610, such as a magnetic disk, optical disk, or a Solid State Drive (SSD) is provided and coupled to bus 602 for storing information and instructions.

Computer system 600 may be coupled via bus 602 to a display 612, such as a cathode ray tube (CRT), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane.

Computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor 604 executing one or more sequences of one or more instructions contained in main memory 606. Such instructions may be read into main memory 606 from another storage medium, such as storage device 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 610. Volatile media includes dynamic memory, such as main memory 606. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface 618 coupled to bus 602. Communication interface 618 provides a two-way data communication coupling to a network link 620 that is connected to a local network 622. For example, communication interface 618 may be an integrated services digital network (ISDN) card, cable modem, satellite modem, or a modem to provide a data communication connection to a corresponding type of telephone line. As another example, communication interface 618 may be a local area network (LAN) card to provide a data communication connection to a compatible LAN. Wireless links may also be implemented. In any such implementation, communication interface 618 sends and receives electrical, electromagnetic or optical signals that carry digital data streams representing various types of information.

Network link 620 typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the world wide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are example forms of transmission media.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618.

The received code may be executed by processor 604 as it is received, and/or stored in storage device 610, or other non-volatile storage for later execution.

### 11. MISCELLANEOUS; EXTENSIONS

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected, and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
accessing a plurality of clinical guideline vector embeddings associated with a plurality of clinical guidelines;
receiving a request query for a medical procedure for a patient;
generating a query vector embedding for the request query by applying a vector embedding function to the request query;
calculating similarity scores between the query vector embedding and the each of the plurality of clinical guideline vector embeddings;
identifying a subset of clinical guideline vector embeddings having a similarity score with the query vector embedding above a threshold value;
generating a prompt for providing to a generative AI model for generating a prior authorization report;
providing the prompt to the generative AI model;
applying the generative AI model to the subset of clinical guidelines and patient data to determine a target clinical guideline and generate a prior authorization report responsive to the prompt, wherein the patient data comprises the request query, health plan details, and patient details;
based on the target clinical guideline and the patient data, generating, by the generative AI model, a prior authorization report responsive to the prompt;
wherein the patient is treated based on an approval of the medical procedure in the prior authorization report.

2. The one or more non-transitory computer readable media of claim 1, wherein the prompt comprises one or more of:
i. a role assignment;
ii. inputs;
iii. an output format; or
iv. reasoning steps.

3. The one or more non-transitory computer readable media of claim 2, wherein the role assignment directs the generative AI model to respond in a role of a medical expert evaluating requests for appropriateness and cost-effectiveness.

4. The one or more non-transitory computer readable media of claim 2, wherein inputs comprise, one or more of: (a) clinical notes, (b) family history, (c) diagnostic results, (d) procedure request, (e) health plan detail, (f) therapy description, (g) disease description, (h) supporting documents, or (i) location of service.

5. The one or more non-transitory computer readable media of claim 2, wherein the output format comprises one or more of:
i. a decision comprising at least one of: (a) approved, (b) step therapy, (c) alternative treatment, or (d) denied;
ii. a table comprising at least one of: (a) symptom summary, (b) guideline alignment, (c) health plan coverage, or (d) service location;
iii. a recommendation; or,
iv. a guideline source.

6. The one or more non-transitory computer readable media of claim 2, wherein the reasoning steps comprise one or more of:
i. matching symptoms to guidelines,
ii. evaluating procedure appropriateness; or
iii. verifying help plan coverage and alignment.

7. The one or more non-transitory computer readable media of claim 1, wherein accessing a plurality of clinical guideline vector embeddings comprises:
accessing the plurality of clinical guidelines, the clinical guidelines of the
plurality of clinical guidelines comprising headings and subheadings;
parsing the clinical guidelines of the plurality clinical guidelines to identify the headings and the subheadings;
segmenting the clinical guidelines into contextual chunks based on the headings and the subheadings;
generating the plurality of clinical guideline vector embeddings by applying a vector embedding function to each of the contextual chunks of the plurality of clinical guidelines; and
indexing the plurality of clinical guideline vector embeddings.

8. The one or more non-transitory computer readable media of claim 1, wherein the request query comprises: (a) a procedure request, (b) a therapy description, (c) a disease description, and (d) a location of service.

9. The one or more non-transitory computer readable media of claim 1, wherein the operations further comprise:
reranking the subset of clinical guideline vector embeddings to generate a ranked subset of vector embeddings,
wherein a target clinical guideline vector embedding is selected from the ranked subset of clinical guideline vector embeddings.

10. The one or more non-transitory computer readable media of claim 1, wherein the operations further comprise:
performing a k-nearest neighbor search against the plurality of clinical guidelines vector embeddings to identify a top k clinical guideline vector embeddings most similar to the query vector embedding;
wherein calculating similarity scores between the query vector embedding and the each of the plurality of clinical guideline vector embeddings comprises:
calculating the similarity score between each of the top k clinical guideline vector embeddings and the query vector embedding.

11. A method comprising:
accessing a plurality of clinical guideline vector embeddings associated with a plurality of clinical guidelines;
creating a request query for a medical procedure for a patient;
generating a query vector embedding for the request query by applying a vector embedding function to the request query;
calculating similarity scores between the query vector embedding and the each of the plurality of clinical guideline vector embeddings;
identifying a subset of clinical guideline vector embeddings having a similarity score with the query vector embedding above a threshold value, wherein the subset of clinical guideline vector embeddings correspond to a subset of clinical guidelines; and
generating a prompt for providing to a generative AI model for generating a prior authorization report;
providing the prompt to the generative AI model;
applying the generative AI model to the subset of clinical guidelines and patient data to determine a target clinical guideline and generate a prior authorization report responsive to the prompt, wherein the patient data comprises the request query, health plan details, and patient details; and
based on the target clinical guideline and the patient data, generating, by the generative AI model, a prior authorization report responsive to the prompt;
wherein the patient is treated based on an approval of the medical procedure in the prior authorization report,
wherein the method is performed by at least one device including a hardware processor.

12. The method of claim 11, wherein the prompt comprises a role assignment wherein the role assignment directs the generative AI model to respond in a role of a medical expert evaluating requests for appropriateness and cost-effectiveness.

13. The method of claim 11, wherein the prompt comprise inputs including one or more of: (a) clinical notes, (b) family history, (c) diagnostic results, (d) procedure request, (e) health plan detail, (f) therapy description, (g) disease description, (h) supporting documents, or (i) location of service.

14. The method of claim 11, wherein the prompt comprises an output format, the output format comprising one or more of:
i. a decision comprising at least one of: (a) approved, (b) step therapy, (c) alternative treatment, or (d) denied;
ii. a table comprising at least one of: (a) symptom summary, (b) guideline alignment, (c) health plan coverage, or (d) service location;
iii. a recommendation; or,
iv. a guideline source.

15. The method of claim 11, wherein the prompt comprises a reasoning steps, the reasoning step comprising one or more of:
i. matching symptoms to guidelines,
ii. evaluating procedure appropriateness; or
iii. verifying help plan coverage and alignment.

16. The method of claim 11, wherein accessing a plurality of clinical guideline vector embeddings comprises:
accessing the plurality of clinical guidelines, the clinical guidelines of the plurality of clinical guidelines comprising headings and subheadings;
parsing the clinical guidelines of the plurality clinical guidelines to identify the headings and the subheadings;
segmenting the clinical guidelines into contextual chunks based on the headings and the subheadings;
generating the plurality of clinical guideline vector embeddings by applying a vector embedding function to each of the contextual chunks of the plurality of clinical guidelines; and
indexing the plurality of clinical guideline vector embeddings.

17. The method of claim 11, wherein the request query comprises: (a) a procedure request, (b) a therapy description, (c) a disease description, and (d) a location of service.

18. The method of claim 11, further comprising:
reranking the subset of clinical guideline vector embeddings to generate a ranked subset of vector embeddings,
wherein a target clinical guideline vector embedding is selected from the ranked subset of clinical guideline vector embeddings.

19. The method of claim 11, further comprising:
performing a k-nearest neighbor search against the plurality of clinical guidelines vector embeddings to identify a top k clinical guideline vector embeddings most similar to the query vector embedding;
wherein calculating similarity scores between the query vector embedding and the each of the plurality of clinical guideline vector embeddings comprises:
calculating the similarity score between each of the top k clinical guideline
vector embeddings and the query vector embedding.

20. A system comprising:
at least one device including a hardware processor;
the system being configured to perform operations comprising:
accessing a plurality of clinical guideline vector embeddings associated with a plurality of clinical guidelines;
creating a request query for a medical procedure for a patient;
generating a query vector embedding for the request query by applying a vector embedding function to the request query;
calculating similarity scores between the query vector embedding and the each of the plurality of clinical guideline vector embeddings;
identifying a subset of clinical guideline vector embeddings having a similarity score with the query vector embedding above a threshold value, wherein the subset of clinical guideline vector embeddings correspond to a subset of clinical guidelines; and
generating a prompt for providing to a generative AI model for generating a prior authorization report;
providing the prompt to the generative AI model;
applying the generative AI model to the subset of clinical guidelines and patient data to determine a target clinical guideline and generate a prior authorization report responsive to the prompt, wherein the patient data comprises the request query, health plan details, and patient details;
based on the target clinical guideline and the patient data, generating, by the generative AI model, a prior authorization report responsive to the prompt;
wherein the patient is treated based on an approval of the medical procedure in the prior authorization report.
